(19) United States
(12) Patent Application Publication
(10) Pub. No.: US 2009/0299329 A1
(43) Pub. Date: Dec. 3, 2009

(54) COLD TREATMENT

(76) Inventor: Charles Richard DIXON, Warwickshire (GB)

Correspondence Address: NIXON PEABODY, LLP, 401 9TH STREET, NW, SUITE 900, WASHINGTON, DC, 20004-2128, US

(21) Appl. No.: 12/474,509

(22) Filed: May 29, 2009

Related U.S. Application Data
(60) Provisional application No. 61/057,379, filed on May 30, 2008.

(51) Int. Cl. A61M 31/00

(52) U.S. Cl. 604/514

(57) ABSTRACT

The invention relates to a method of reducing or ameliorating one or more symptoms of a cold or flu in a subject, comprising the steps of providing a source of water vapour, manipulating the subject's nose to occlude one nasal passageway, inhaling the water vapour through the remaining nasal passageway, and exhaling.

### RELATED APPLICATION

This application claims the benefit of U.S. Provisional Patent Application Ser. No. 61/057,379, filed May 30, 2008.

### FIELD OF THE INVENTION

The present invention relates generally to the field of treating an infectious ailment, such as the common cold. More particularly, the present invention relates to a method of reducing or ameliorating one more symptoms of a cold or a flu in a subject.

### BACKGROUND TO THE INVENTION

Common colds and other acute respiratory infections are the most frequent acute illnesses, and contribute a significant proportion to all the time lost from employees and absenteeism from school. Generally, while suffering from the common cold, the nasal passages of a subject's nose tend to be obstructed. Passages may be heavily obstructed by mucus residing therein. This obstruction in the nasal passages can cause the subject discomfort.

A technique that has been employed in order to alleviate the symptoms caused by the common cold and other acute respiratory infections has been to inhale warm damp air with the intention of making the mucus inside the body drain away more easily.

Numerous devices have been developed over the years in order to provide a means for generating a stream of water vapour/steam for inhalation by a subject. One particular device, the RHINOTHERM ULTRA® (Ultra Rhino-Therm, Ltd., Israel), functions by providing a dual jet mechanism which propels humidified air towards the nasal passages of a subject.

### BRIEF DESCRIPTION OF THE DRAWINGS

The drawings are provided to assist understanding of the invention and should be interpreted with this in mind. Drawings are not be regarded as limiting the scope of the claims.

FIG. 1a is a front elevation of a subject conducting the treatment in accordance with one embodiment of the present invention.

FIG. 1b is a front elevation of a subject as shown in FIG. 1a, but of an alternative embodiment of the present invention.

FIG. 2 is a front elevation similar to that shown in FIG. 1a, but of an alternative embodiment of the present invention.

### DESCRIPTION OF THE INVENTION

Although the present invention will hereinafter be described with particular reference to its application in treating the common cold, it will be appreciated that the present invention may be utilised to treat other ailments with which a person skilled in the art would be familiar. It will be understood, however, that this description is provided without prejudice to the generality of the invention or its range of applications. In particular, the invention may be applied to any disorder, such as infectious disorder, where the infectious pathogen (e.g., virus or bacterium) colonises or accumulates in the nasal passage, or triggers an allergic or other immune response that occludes the nasal passages. Examples of such disorders include those caused by picornaviruses, coronaviruses, parainfluenza viruses, adenoviruses, influenza viruses, or other similar viruses, particularly those transmitted by aerosol. Most suitably the invention is applied to the common cold, such as caused by rhinoviruses.

From the discussion that is to follow, it will become apparent how the present invention addresses difficulties and problems associated with the prior art whilst providing numerous additional advantages not hitherto contemplated in prior art techniques.

According to an aspect of the present invention, there is provided a method of reducing or ameliorating one or more symptoms of a cold or flu in a subject, comprising the steps of:

(a) providing a source of water vapour;

(b) manipulating the subject's nose to occlude one nasal passageway;

(c) inhaling said water vapour through the remaining nasal passageway; and

(d) exhaling.

The method enables a subject to target one nasal passageway at a time. Where one nasal passageway is obstructed to a greater extent than the other, inhalation through both nostrils concurrently can be uncomfortable by way of causing pain in the subject's paranasal sinuses. Without wishing to be bound by theory, by manipulating the subject's nose so as to occlude one nasal passageway allows the remaining nasal passageway to be targeted thus reducing the likelihood of pain in the sinal regions of the subject's face.

A further advantage offered by the method of the present invention is that by manipulation of the subject's nose to occlude one nasal passageway creates a vacuum which allows the subject to inhale with greater force than if both nasal passageways were unobstructed, in that the suction is improved. Improvement of the force by which the subject is able to inhale facilitates unobstruction of the nasal passageway and reduction or amelioration of one or more symptoms of a cold in the subject.

Furthermore, in any one subject the maximum lung capacity is generally fixed. Therefore, by utilising the method of the present invention and inhaling water vapour through only one nasal passageway at a time, as opposed to both nasal passageways associated with the prior art techniques, reduces the overall water vapour inlet size due to the occlusion of one nostril. Thus, it would be expected that a subject would be required to inhale for approximately twice the length of time to reach maximum lung capacity than that which it would take if both nasal passageways were utilised during inhalation. This is because the same amount of water vapour is being inhaled by the subject, but through a smaller water vapour inlet (one nasal passageway as opposed to two nasal passageways). The method, therefore, concentrates the water vapour in the selected nasal passageway because the water vapour passes through the remaining nasal passageway for a duration of time than if both nasal passageways were employed during inhalation. As a result, the method provides a more efficient and robust technique for reducing or ameliorating one or more symptoms of a cold in a subject than those provided by known techniques.

It is an advantage of the present invention that when working the method, a greater intranasal temperature may be achieved in the subject's nasal cavity. This is due to it taking approximately twice the length of time to fill the subject's lungs via a single nasal passageway than it would to fill those same lungs via both nasal passageways as in known methods. This approximate doubling of the amount of time taken to fill the lungs means that the nasal cavities are treated for approximately twice the length of time per cycle than in known methods. This allows the therapeutic intranasal temperature elevation to be sustained for longer per cycle than with known methods. Furthermore, this is also sustained for a time greater than the cumulative time which would be achieved by repeating a prior art cycle, since transit time and temperature drop by exhalation is advantageously avoided in each cycle compared to known techniques.

It is an advantage of the present invention that greater consistency of intranasal temperature is achieved. For a given lung volume, if dual passageway inhalation is used as according to known methods, there will inevitably be a cycling or a dropping of the temperature during the exhalation phase. An advantage of the present invention is that the higher temperature is sustained throughout the inhalation phase of the cycle, providing greater consistency to the method.

It is an advantage of the invention that, as noted above, by performing the method as described a greater time is spent with the nasal passageways at the optimal subscalding temperature. This is because, for the same inhalation volume, practicing according to the present invention results in the intranasal temperature being maintained at the optimum elevated temperature for longer per lung volume than with prior art methods.

Due to the greater consistency and greater time spent at the elevated intranasal temperature, a greater carry of the water vapour (and/or biocidals if used) is achieved. This greater efficiency of transport leads to a correspondingly higher delivery of the vapour and/or biocidals to the subject's nasal cavity.

It should also be noted that the present invention provides a greater consistency in terms of the overall treatment to the nasal passages. For example, according to known techniques, it is most unusual for each nostril or nasal passageway to provide an equivalent for resistance or and equivalent flow. If one passageway is partially blocked, an inequality develops. Depending on the severity of the blockage, inhalation via dual passageway techniques may lead to a 60/40 split of treatment, or an 80/20 split or even worse, if one passageway is barely permitting any flow at all the difference can be exaggerated even further. By practicing according to the present invention, regardless of the actual flow rate per passageway, each passageway can be systematically subjected to the same flow volume. This has the advantage of evenly distributing the treatment throughout each nasal passageway in turn. Although with a lower flow rate which might be achieved due to the partial blocking of one nasal cavity, it is important to note that the total treatment i.e. the total flow volume taken in per passageway in each cycle will be equalised. This advantageously evens out the delivery of water vapour (and/or biocidal) to each passageway in turn. Furthermore, it helps to ensure that the optimal subscalding intranasal temperature is achieved in turn in each nasal passageway. Again, according to known techniques, an inequality of flow between the dual nasal passageways which are used simultaneously would inevitably lead to an inequality of temperatures generated in each nasal cavity. For example, according to known techniques, the subject is likely to reduce the water vapour temperature when one nasal passageway reaches the optimal subscalding temperature. This is likely to leave the partially blocked passageway to never achieve the optimum temperature since to increase the flow in the partially blocked passageway or to increase the temperature of the water vapour to achieve the correct optimal treatment temperature inside that blocked passageway is to risk scalding the free flowing passageway. These problems are advantageously avoided by practicing according to the present invention i.e. by practicing occlusion of individual nasal passageways in turn as taught herein.

Without wishing to be bound by theory, it is believed that the methods of the invention are effective in the destruction or inactivation of pathogens such as rhinoviruses capable of causing the common cold. In particular, these pathogens are known to accumulate and possibly to multiply in the nasal passages before a full blown disease develops in the subject. It is an advantage of practicing the methods of the invention that the pathogens are killed or inactivated at a much earlier stage in the course of the disease than with known methods. Indeed, it is possible that persistent or extended periods of disease can be caused by reinfection from a pool of pathogen particles which have collected or accumulated in the subjects nasal passageways. Again, it is an advantage of the invention that this phenomenon is addressed and ameliorated.

As explained above, the treatments disclosed herein kill off the infection at a very early stage. This stage can be so early as to be before the subject's immune system has mounted a developed response against the pathogen such as this specific virus. In particular, the treatments of the invention can address the infection before the subject's immune system has developed antibodies to the particular strain of pathogen present. Thus, in order to combat the risk of reinfection inherent with this treatment, the method is advantageously repeated until symptoms have completely disappeared. Advantageously, those with whom the subject may come into contact such as family members, work colleagues or other individuals should also undertake the treatment in order to further minimise the chance of reinfection.

The invention is suitably applied to colds such as the common cold. The techniques can enjoy up to about 80% success in addressing colds, or even more. When applied to flu, the invention may best be used in addressing symptoms of the disease which are in common with symptoms of a cold such as runny nose and/or sneezing. Because influenza (‘flu’) is a different disorder caused by a different pathogen, the invention may not be as effective in treatment of flu as it is in treatment of colds and for these reasons the methods are typically used to treat colds as the principal application of the invention.

The source of water vapour may be provided by boiled water. Boiled water may be superior to actively boiling water because the water temperature can be controlled more easily. Furthermore, the temperature of the water vapour can be below 100° C. This is beneficial because it may help to prevent harm caused to the subject by reducing the opportunity for the water vapour to reach a temperature which would cause harm to the subject. A further advantage of boiled water is that it is substantially sterile due to the boiling step.

The source of water vapour (such as boiled water) may be located in a container. The container may have a substantially upright wall. The container may be cylindrically shaped. The container may have a substantially flat base to provide a stable footing in order to reduce the risk of spillage of the source of water vapour (such as boiled water), which could cause harm to the subject. The subject may position his/her face above the source of water vapour so that the water vapour may be readily inhaled as it rises.

The step of exhaling in the method may be performed through the subject's mouth. The mouth, being a significantly larger opening than a nostril, allows speedy expulsion of water vapour when required.

Alternatively or additionally, the step of exhaling may be performed through at least one of the subject's nasal passageways. More particularly, the exhalation step may occur through the same nasal passageway used for the step of inhaling. This may help dislodge any mucous residing in a subject's nasal passageway because water vapour would be conveyed in both directions through the said passageway. Furthermore, this advantageously passes the water vapour back through the nasal passageway a second time during exhalation. Thus, this embodiment has a further advantage of approximately doubling the amount of exposure of the nasal passage to the vapour for each inhalation-exhalation cycle.

Steps c) to d) of the method, more specifically the inhaling and exhaling steps respectively, may be repeated at least three times. This may help to ensure that the symptoms of a cold are reduced to the fullest extent possible, and to help reduce the likelihood of the symptoms reappearing immediately. Similarly, steps c) to d) may be repeated for a period of at least five minutes to provide the same advantage. If required, steps c) to d) may be repeated for at least a further three minutes.

The method may comprise the step of providing means for retaining the water vapour in an enclosed space for inhaling. The water vapour may then readily be inhaled by the subject in an efficient manner. If the water vapour is enclosed it may be utilised for a longer period than would otherwise be possible without the means for retaining the water vapour. Thus, this embodiment has the further advantage of preventing or slowing dispersal of the water vapour into the atmosphere.

The means for retaining the water vapour in an enclosed space may be a fabric draped over the subject's head and the source of water vapour. The fabric may comprise as a substantially impermeable layer capable of retaining the water vapour in the desired space. Use of a fabric may be advantageous because it can be quickly removed from the subject's head should the temperature of the water vapour exceed a preselected comfort threshold.

Further regarding temperature, the temperature of the water vapour is important. The water vapour is occasionally referred to as “warm damp air”. More correctly, this will be referred to as water vapour from this point on. For the purposes of the invention, water vapour means air comprising droplets of condensed water. This is often referred to in common parlance as “steam”. Of course, in a strict chemical sense, steam is a colourless gas comprising 100% atomised water (H₂O). At normal atmospheric pressure, “steam” will only persist at a temperature of at least 100° C. For the purposes of the present invention, the colloquial use of the word “steam” is preferred, i.e., reference to the cloudy grey/white visible condensing water vapour is the “water vapour” of the present invention. This is important because the water vapour is inhaled through the subject's nasal passageway, and therefore the temperature needs to be such as to avoid scalding.

The temperature of the water vapour is suitably about 40° C. to 60° C., more suitably about 40° C. to 55° C., 43° C. to 50° C., 43° C. to 49° C., 45° C. to 47° C., or about 47° C. The temperature of the water vapour is suitably the temperature at the point of inhalation.

Determining the temperature of the water vapour at the point of inhalation can be technically demanding. Alternative guidance is that the water vapour/air should feel hot enough to make the subjects face perspire, but not so hot as to scald the inside of the subjects nose.

Clearly, the subject practicing the invention can take steps to vary the temperature of the water vapour at the point of inhalation, for example to reduce the temperature the subject may move slightly further away from the source of the water vapour, and conversely in order to increase the temperature the subject may move closer to the source of water vapour. If the source of water vapour is or comprises boiled water, as this water cools the useful temperature range of the water can be preserved or extended by breathing out gently onto the surface of the water in order to force further warm damp air/water vapour off the surface which can then be inhaled. Typically, one conventional mug full of boiling water should provide a source of water vapour sufficient for a complete treatment.

Due to the possible technical challenges in measuring the temperature of the water vapour at the point of inhalation, when the source of water vapour comprises a source of hot water, the temperature of that water may be used as a guide to the temperature of the water vapour generated. In this regard, typically the temperature of the water which is used as the source of water vapour should be in excess of 50° C. for optimum results. The water vapour may have a temperature of at least about 47° C. This may be an optimal temperature in order for the method to be conducted with efficiency.

Further, regarding timing and duration. each single treatment is suitably approximately 5 minutes. Also suitably, each single treatment is about 8 minutes to 10 minutes. These timings include conducting the method through each nasal passageway independently, therefore the time per each individual nasal passageway is approximately half the total stated time for conducting the method.

The method may be conducted for a period ranging from 8 minutes to 10 minutes. This may be an optimal duration of time for which the method should be performed.

In order to be maximally effective, the method may be commenced promptly following observation of symptoms. Suitably the method may be conducted within 36 hours of the onset of symptoms. For example, the method should be commenced within about twelve hours of the onset of symptoms. Alternatively, the method may be commenced within about seven hours of onset of symptoms, such as within about five hours of the onset of symptoms. Indeed, the method may be commenced within about three hours of the onset of symptoms, such as within about two hours of the onset of symptoms. If possible, the method may be commenced within about one hour of the onset of symptoms, or immediately following the onset of symptoms.

The subject's nose may be manipulated by pressing the side of the nose with the subject's finger. The subject's finger is able to adapt easily to the contours of the subject's nose so that it may be held in the desired manipulated form for the duration of the treatment. Should the temperature of the water vapour become too hot for the subject, the subject's hand, to which the pressing finger belongs, may easily be used to act as a shield to prevent the subject's face from being harmed. Alternatively, said hand may be used as a funnel or guide to facilitate inhalation of the vapour.

In the instance where the subject is elderly, or a child, or is otherwise unable to press the subject's nose, another individual may assist in manipulating the subject's nose. Alternatively, any suitable device may be employed for nose manipulation.

The method of the present invention may be employed to target each nostril consecutively, i.e., each nostril in turn. More particularly, steps c) to d) of the method may be repeated after releasing the first occluded nasal passageway and proceeding to occlude the second nasal passageway. Concentrating the water vapour on each nostril independently improves the efficacy of the method. Manipulating the nose from either side to target one nostril at a time also ensures that the subject breathes through both nostrils, which does not occur in known techniques.

Symptoms of a cold (or of a flu) include one or more of runny nose, headache, sore throat, coughing, sneezing, congestion or other symptoms known to those skilled in the art.

Advantageously, the treatment should be commenced promptly following the observation of symptoms of a cold. E.g., within about five hours, within about four hours, within about three hours, within about two hours, within about one hour, or immediately after observing the first symptom(s).

In a one embodiment, the source of water vapour comprises boiled water. This may or may not comprise additional components. For example, if desired, biocidal ingredients such as friar's balsam or other conventional additives may be comprised by the source of water vapour.

Suitably the ailment is a cold such as a common cold, e.g., as caused by a relevant rhinovirus.

Various embodiments of the present invention will now be described more particularly by way of example, with reference to, the accompanying drawings, in which:

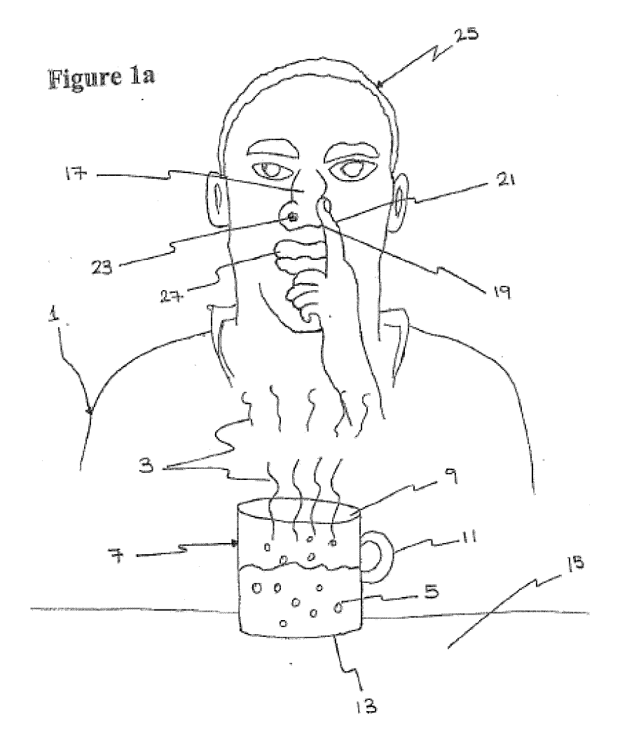

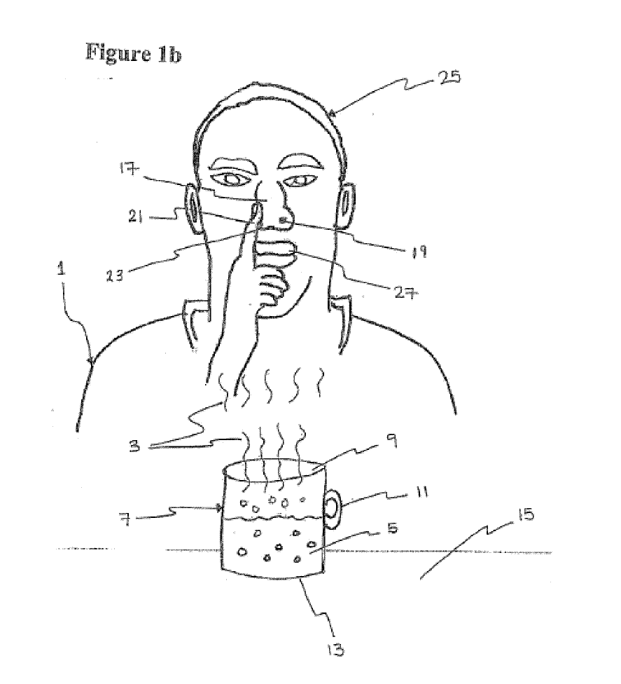

FIG. 1a is a front elevation of a subject conducting the treatment in accordance with one embodiment of the present invention;

FIG. 1b is a front elevation of a subject as shown in FIG. 1a, but of an alternative embodiment of the present invention; and

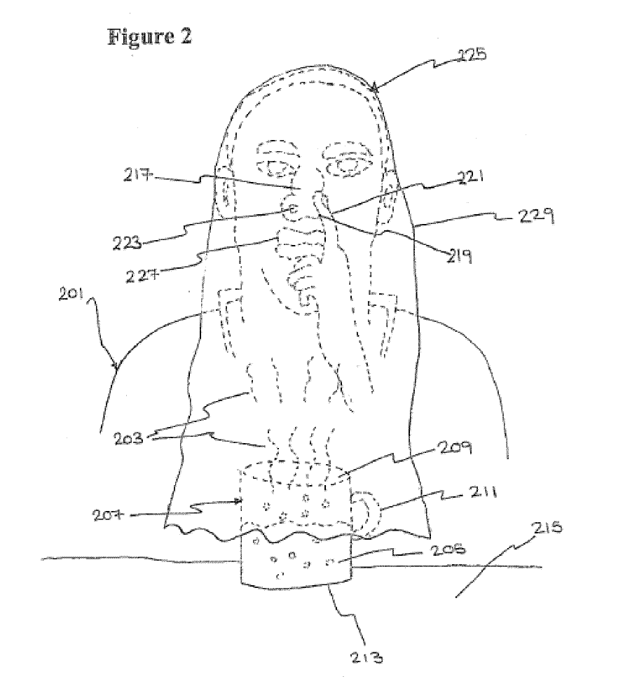

FIG. 2 is a front elevation similar to that shown in FIG. 1a, but of an alternative embodiment of the present invention.

It should be noted that the figures show the distance between the head and receptacle as being relatively large. In practicing the invention this distance should be varied by the user in accordance with the guidance given herein, e.g., to achieve optimal sub-scalding water vapour temperatures. This will typically mean that the distance between the head and receptacle will be smaller than is represented in the drawings. The drawings are provided to assist understanding of the invention and should be interpreted with this in mind, and should not be regarded as limiting the scope of the claims.

Referring first to FIG. 1a, there is shown a subject 1 performing an embodiment of the method in accordance with the present invention so as to reduce or ameliorate one or more symptoms of a common cold. In this embodiment, the subject 1 is suffering from a common cold. There is provided a source of water vapour 3 in the form of boiled water 5. The boiled water 5 resides in a cylindrically-shaped mug 7. The mug 7 has a volume of 568 ml and is approximately half filled with boiled water 5. Water vapour 3 escapes from the open top 9 of the mug 7. The mug 7 also comprises a handle 11 for ease of carrying, and a substantially flat base 13 which provides stability in its resting position. The base 13 rests upon a table 15 having a substantially flat horizontal surface.

The subject 1 is shown to be manipulating his nose 17 so as to occlude one nasal passageway 19 (covered by finger 21 in drawing). Although it will be appreciated that a subject's nose may be manipulated by various means, for example by using a nose peg, foam plug, tissue paper bung or any other suitable means in order to occlude one nasal passageway, in this embodiment the subject is shown to be pressing his finger 21 against the side of his nose 17 so as to occlude the nasal passageway 19. The remaining passageway 23 is partially obstructed by mucus residing therein (not shown), the mucus being caused by the common cold.

The subject's head 25 is positioned above the open top 9 of the mug 7 so as to receive the rising water vapour 3. The subject 1 then inhales the water vapour 3 through the remaining nasal passageway 23 in order to reduce one or more symptoms of the cold. The water vapour 3, at the point it reaches the subject's nose 17, has a temperature of approximately 47° C. It is possible to reduce the temperature of the water vapour 3, at the point it reaches the subject's nose 17, by moving further away from the boiled water 5.

As a final step, the subject 1 exhales. The exhalation occurs via the remaining nasal passageway 23, although it will be understood that in other embodiments exhalation could occur through the other nasal passageway 19 or the mouth 27. Exhalation through the mouth 27 is particularly useful when inhalation or exhalation through either of the nasal passageways 19, 23 becomes difficult—that is, if any residing mucus in the nasal passageway causes an obstruction increasing the air resistance.

The inhalation and exhalation steps are repeated at least three times over the course of five minutes. These steps can be repeated for a further three minutes to ensure that the symptoms do not return immediately.

Having completed the treatment on one side, the opposite nasal passageway 21 can then be targeted. This is achieved by releasing the first occluded nasal passageway 19 by transferring the finger 21 (or using a finger from the other hand) to the opposite side of the nose 17 thereby occluding the second nasal passageway 23, as depicted in FIG. 1b. The inhalation and exhalation steps may then be conducted utilising the second nasal passageway 19.

Referring now to FIG. 2, there is shown an alternative embodiment of the present invention. The illustration is substantially similar to that shown in FIG. 1, thus like reference numerals represent like features, except that a fabric 229 is draped over the subject's head 225 and partially over the mug 207. The fabric 229 acts as a means for retaining water vapour 203 within an enclosed space (between subject's nose 217 and the open end 209 of the mug 207). In this way, inhalation is made easier due to the water vapour 203 being concentrated within an enclosed region beneath the fabric 229. The water vapour 203 may, therefore, retain its temperature for longer durations, which improves the efficiency of the method since longer periods may elapse until the water 205 requires reheating or replacing.

### EXAMPLES

### Example 1

### Subject Has a Cold With a Runny Nose or Sneezing

A first symptom is observed. In this example, the symptom is a runny nose. Alternatively, the symptom may be sneezing. The method is commenced within 12 hours of the first symptom being observed.

Enough water to fill a mug is boiled. A mug is provided and is filled with the boiled water promptly after boiling. In this example, the mug has a base the same size as the top and is straight sided.

The subject thoroughly blows the nose.

The mug of boiled water is placed on a firm level base. The subject positions their face comfortably near to the mug and above the mug. The mug of boiled water is the source of water vapour in this example.

A first nostril is occluded or closed by pressing on that side of a subjects nose with a finger.

The warm damp air (water vapour) from the mug of boiling water is inhaled through the open nostril. The water vapour is approximately 45° C. to 49° C.

The subject breathes in and out three times through the first nostril. This nostril is then released, the opposite nostril is occluded, and the subject breathes in and out three times through said second nostril.

It should be noted that even after blowing the nose, it is often the case that one nostril may be relatively clear and easy to breathe through while the other nostril may remain difficult to breathe through. This does not adversely affect performance of the invention, but it is important to persevere and to breathe in and out through the partially blocked nostril the same number of times as through the clear nostril. If this is particularly difficult, exhalation may be conducted through the mouth in order to ease performance of the method.

The method should be conducted until cold symptoms disappear. In this example this happened at about 5 minutes.

Optionally the method is continued for a further 3 minutes. In this example, the method was continued for approximately 3 further minutes.

The method should be repeated if symptoms reappear.

### Example 2

### Common Colds Experienced by a Subject on Different Occasions

Each of the colds which will be described hereinafter were experienced by a subject over the course of three winter periods. These were cured according to the invention as follows.

Winter One, Cold One: The subject observed symptoms of a cold, including severe sneezing and a runny nose, which began suddenly. The treatment in accordance with the present invention was initiated within 5 minutes of observing the cold symptoms. Following the treatment, two hours later the symptoms reappeared. The subject repeated the steps of the method and the symptoms disappeared. Two hours later, the symptoms again reappeared. The subject repeated the steps of the method for a final time but continued for about three minutes after the symptoms disappeared, not to return.

Winter One, Cold Two: The subject experienced severe sneezing and a runny nose. The treatment was commenced within five minutes of observing the cold symptoms, following which the symptoms disappeared. The same variation of the treatment was employed as the successful attempt with the previous cold. The symptoms did not reappear.

Winter Two, Cold Three: The subject became aware that he was suffering from a common cold during the evening. The subject had disregarded an occasional sneeze earlier that day as being attributable to a mild allergy, but later concluded that the sneezing had been the start of a common cold, which had taken effect ten hours previously. As soon as realising this, the subject began the treatment immediately in accordance with the steps of the method. The symptoms did not clear after the initial attempt of the treatment because breathing in through one of the nasal passageways had become extremely difficult due to the accumulation of mucus therein. The subject waited for a further 15 minutes to allow the mucus to disperse before repeating the treatment procedure, following which the symptoms completely cleared and did not return.

Winter Two, Cold Four: This cold occurred a couple of months after cold three and was dealt with in exactly the same manner as cold two.

Winter Three, Colds Five and Six: These were successfully dealt with as colds two and four.

Cold Seven: Friday, 28 Sep. 2007: The subject observed the symptoms of a cold and began the treatment in accordance with the method approximately two hours after said observation. A single treatment was successful and the cold symptoms disappeared, not to return until Monday.

Monday, 1 Oct. 2007: The subject observed symptoms of a cold that had commenced at 2 pm. The symptoms included sneezing and sniffling. The treatment was initiated at 7 pm and was again successful.

Tuesday, 2 Oct. 2007: The symptoms reoccurred in the morning and treatment was begun at 7 pm that evening. Following the treatment, the subject experienced no further symptoms and was able to breathe easily through the nasal passages overnight.

Wednesday, 3 Oct. 2007: The subject awoke with no symptoms and then showered in a cold bathroom. The subject experienced nasal discomfort during showering which suggested that the symptoms were reoccurring. By 11 am that morning the symptoms had returned. The treatment was initiated using a mug of tea as a source of water vapour. Use of the tea as a source of water vapour reduced the symptoms, although repeat treatment was indicated since it did not completely relieve the subject of the symptoms. That evening, the subject repeated the treatment by using boiled water which appeared to eliminate the symptoms.

Thursday, 4 Oct. 2007: The symptoms reappeared in the morning and were treated in the evening—no symptoms were experienced overnight.

Friday, 5 Oct. 2007: During the morning the cold appeared to be moving into its final stages, a few heavy sneezes, but beginning to dry up. The heaviest sneeze was enough to cause a slight pain in the subject's sinuses. The subject was able to breathe easily overnight and no further symptoms were experienced over the whole weekend.

1. A method of reducing or ameliorating one or more symptoms of a cold or flu in a subject, comprising the steps of:
(a) providing a source of water vapour;
(b) manipulating the subject's nose to occlude one nasal passageway;
(c) inhaling said water vapour through the remaining nasal passageway; and
(d) exhaling.

2. The method of claim 1, wherein the source of water vapour is provided by boiled water.

3. The method of claim 1, wherein the step of exhaling is performed through the subject's mouth.

4. The method of claim 1, wherein the step of exhaling is performed through at least one of the subject's nasal passageways.

5. The method of claim 1, wherein the step of exhaling is performed through the same nasal passageway used for the step of inhaling.

6. The method of claim 1, wherein steps c) to d) are repeated at least three times.

7. The method of claim 1, wherein steps c) to d) are repeated for at least five minutes.

8. The method of claim 7, wherein steps c) to d) are repeated for at least a further three minutes.

9. The method of claim 1, further comprising the step of providing means for retaining said water vapour in an enclosed space for inhaling.

10. The method of claim 9, wherein the means for retaining said water vapour in an enclosed space is a fabric draped over the subject's head and the source of water vapour.

11. The method of claim 1, wherein the water vapour has a temperature of at least about 47° C.

12. The method of claim 1, wherein steps c) to d) are performed within a period ranging from about eight minutes to about ten minutes.

13. The method of claim 1, wherein the subject's nose is manipulated by pressing the side of said nose with the subject's finger.

14. The method of claim 1, wherein steps c) to d) are repeated after releasing the first occluded nasal passageway and proceeding to occlude the second nasal passageway.

15. The method of claim 1, wherein said method is commenced within about two hours of onset of one or more symptoms.
